(19) United States
(12) Patent Application Publication
(10) Pub. No.: US 2009/0069343 A1
(43) Pub. Date: Mar. 12, 2009

(54) Combination Histamine H1R and H4R Antagonist Therapy for Treating Pruritus

(76) Inventors: Paul J. Dunford, Solana Beach, CA (US); Lars Karlsson, San Diego, CA (US); Robin L. Thurmond, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/226,309

(22) PCT Filed: Apr. 9, 2007
(86) PCT No.: PCT/US2007/008904

§ 371 Date: Oct. 9, 2008

Related U.S. Application Data
(60) Provisional application No. 60/790,633, filed on Apr. 10, 2006.

(51) Int. Cl. A61K 31/496; A61K 31/135

(52) U.S. Cl. 514/254.08; 514/648

(57) ABSTRACT

Patients suffering from pruritus or itch may be effectively treated by administering a centrally acting histamine H1 receptor antagonist (e.g., diphenhydramine, triprolidine, hydroxyzine, pyrilamine, promethazine, or chlorpheniramine) and a histamine H4 receptor antagonist (e.g., 5-chloro-1H-indol-2-yl)-(4-methyl-piperazin-1-yl)-methanone).

### CROSS-REFERENCE TO RELATED APPLICATION

This application claims priority to U.S. Provisional Application No. 60/790,633, filed Apr. 10, 2006.

### FIELD OF THE INVENTION

The present invention relates to methods and pharmaceutical compositions for treating pruritus combining a centrally acting histamine H1 receptor (H1R) antagonist and a histamine H4 receptor (H4R) modulator.

### BACKGROUND OF THE INVENTION

Pruritus, or itch, is a common and unpleasant symptom of various allergic cutaneous diseases (e.g., atopic dermatitis and hives) and metabolic disorders (e.g., chronic renal failure, hepatic cholestasis, and diabetes mellitus). Although its underlying mechanisms are unclear, histamine is recognized as a mediator of the itch response, eliciting itch when administered to the skin of humans or animals.

The roles of histamine H1, H2, H3, and H4 receptors in acute itch induced by histamine have been investigated. See, e.g., Bell et al., “Involvement of histamine H4 and H1 receptors in scratching induced by histamine receptor agonists in BalbC mice,” British J. Pharmacology (2004), 142(2): 374-380. Additionally, certain histamine H1 receptor antagonists have been mainstay treatments for atopic allergy, but have been found only partially effective in relieving symptoms. The more recent discovery of the fourth histamine receptor, H4R, expressed on hematopoietic cell types often implicated in the development and symptomatology of allergy, has suggested that pharmacological targeting of the H4 receptor in combination with H1 receptor antagonists may prove useful for treating allergy. Fung-Leung et al., “Histamine H4 receptor antagonists: the new antihistamines?”, Current Opinion Invest. Drugs (2004), 5(100): 1174-83. Various combinations of histamine receptor inverse agonists or antagonists have also been described. See, e.g., International Publication Nos. WO 2002/056871 and WO 2004/066960.

Although certain combinations of various histamine receptor antagonists have been described in the literature, there remains a desire for more effective and advantageous therapies for treating pruritus.

### SUMMARY OF THE INVENTION

It has now been discovered that a combination of a histamine H4 receptor antagonist and a centrally acting histamine H1 receptor antagonist has a synergistic or advantageous effect in the treatment of pruritus. Thus, the invention is directed to general and preferred embodiments of methods and pharmaceutical compositions for treating pruritus defined in the appended independent and dependent claims, respectively, which for the sake of brevity are incorporated by reference herein. Additional preferred embodiments, features, and advantages of the invention will be apparent from the following detailed description taken in conjunction with the drawing figures.

### BRIEF DESCRIPTION OF THE DRAWINGS

FIGS. 1-7 depict the results of various comparative studies of the effects of different antihistaminergic agents in an animal model developed to mimic pruritic response (i.e., the itch response mouse model).

FIG. 8 is a bar graph from the results of the itch response mouse model employing histamine to induce itch as described in Example 1a below, showing bouts of scratching for each test agent or control vehicle administered.

FIG. 9 is a bar graph from the results of the itch response mouse model of pruritus employing Compound 48/80 to induce itch as described in Example 1b.

### DETAILED DESCRIPTION OF INVENTION AND ITS PREFERRED EMBODIMENTS

The invention is related to pruritus therapies employing combinations comprising an H4R antagonist with a centrally acting H1R antagonist. The invention may be more fully appreciated by reference to the following detailed description, including the following definitions of certain terms and the concluding examples. For the sake of brevity, the disclosures of the publications, including patents, cited in this specification are herein incorporated by reference.

As used herein, the terms “including”, “containing”, and “comprising” are used herein in their open, non-limiting sense.

The term “subject” refers to a mammalian patient in need of therapeutic or prophylactic treatment. Preferably, subjects treated in accordance with the invention are human.

The term “treat” or “treating” is intended to refer to administration of a composition of the invention to a subject for the therapeutic or prophylactic benefit of reversing, ameliorating, alleviating, inhibiting the progress of, lessening the severity of, or preventing a disease, condition, or disorder, or symptom thereof. In accordance with the invention, effective amounts of a centrally acting histamine H1R antagonist and a histamine H4R antagonist are administered to subjects to treat pruritus.

An “effective amount” means an amount or dose sufficient to generally bring about the desired therapeutic or prophylactic benefit in patients in need of treatment for pruritus. Effective amounts or doses of the antihistamine agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the pruritus or disease, disorder, or condition underlying symptomatic itch, the subject's previous or ongoing therapy, the subject's health status and response to drugs, and the judgment of the treating physician. An exemplary dose is in the range of from about 0.001 to about 200 mg of each antihistamine (H1R or H4R) agent per kg of subject's body weight per 24 hours, preferably about 0.05 to 100 mg/kg/day, or about 1 to 50 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day. Other suitable dosages for antihistamines may be routinely determined in light of the dosages for antihistamines exemplified in the art and in commercial products. See, e.g., Simons et al., New England J. Medicine (1994), 330: 1663-1670.

Once improvement of the pruritus has occurred, the dose may be adjusted for preventative or maintenance treatment, if indicated. For example, the dosage or the frequency of administration, or both, may be reduced as a function of the reduction of itch intensity, to a level at which the desired therapeutic or prophylactic effect is maintained. Of course, if the itch has been alleviated to an appropriate level, treatment may cease. Patients may, however, be intermittently treated on a long-term basis with the antihistaminergic agents of the invention upon any recurrence of pruritus or symptomatic itch.

The antihistaminergic agents of the invention comprise a histamine H1 receptor antagonist that is centrally acting and a histamine H4 receptor antagonist. “Centrally acting” H1R antagonists are H1R antagonists that readily enter the central nervous system (CNS) across the blood-brain barrier (BBB) or have high BBB permeability, and are sometimes referred to in the literature as “classical” histamine H1 receptor antagonists and include a number of first-generation antihistamines. See, e.g., Ishiguro et al., Drug Metabolism & Disposition (2004), 32(5): 519-524; Hansen et al., Drugs Aging (2005), 22(4): 289-96; Welch et al., Clin. Allergy Immunol. (2002), 17: 337-88. Centrally acting H1 receptor antagonists may also include some second- or later-generation antihistamines. See, e.g., Shigemoto, European J. Pharmacology (2004), 494: 161-165.

Preferred centrally acting H1 receptor antagonists include, e.g., d iphen hydra mine, triprolidine, hydroxyzine, pyrilamine, promethazine, chlorpheniramine, and ketotifen, and their pharmaceutically acceptable salts. Other suitable centrally acting H1R antagonists that may be employed in the invention may be selected from lipophilic H1 receptor antagonists and/or through routine testing.

For example, compounds having histamine H1 receptor antagonizing activity that are known or become available may be tested for central activity (i.e., to determine if they are centrally acting) by measuring their ability to cause sedation in humans. An H1R antagonist that is positive in such a screen, i.e., that causes significant sedation in humans, is one that may be selected for use in the invention. Alternatively, to screen H1R antagonists for compounds useful in the invention artisans may directly measure the binding of the antagonist to human brain H1 receptors in vivo using positron emission tomography (PET) (see, e.g., Yanai et al., Br. J. Pharmacol. (1995), 116:1649-1655; Yania et al., J. Neurochem. (1992), 59:128-136; Tagawa et al., Br. J. Clin. Pharmacol. (2001), 52(5): 501; and Tashiro et al., British J. Clin. Pharmacology (2005), 61(1): 16-26). In another technique, this property may be measured in animals, e.g., by quantitating the amount of the H1R antagonistic compound in the brain by LC/MS after oral dosing (Chen et al., Drug Metabolism and Disposition (2003), 31:312-318). In another technique employing animals, the ratio of therapeutic dose to lethal dose in guinea pigs may be used as a measure of central activity, where brain penetration decreases as ratios increase (Van Wauwe et al., Arch. Int. Pharmacodyn. Ther. (1982), 251:39-51). Or H1R antagonists may be assayed for central activity by testing their ability to inhibit the in vitro binding of ³H-mepyramine as described by McQuade et al., Drug Dev. Research (2004), 20(3): 301-06. Another in vitro method for screening for centrally acting compounds is the immobilized artificial membrane phosphatidylcholine column chromatography test described by Yoon et al., J. Biomolecular Screening (2006), 11(1): 13-20. Alternatively, a battery of tests may be employed, such as the subjective sleepiness test (evaluated by Stanford Sleepiness Scale), objective psychomotor test, and measurement of histamine H1-receptor occupancy in the brain as described by Tashiro et al., J. Clinical Pharmacology (2004), 44: 890-900.

Exemplary histamine H4R antagonists that may be used in the methods and compositions of the invention may also be selected from the various compounds known in the art having histamine H4 receptor antagonistic activity. In preferred embodiments, the H4R antagonist of the combination antihistamine therapy of the invention is selected from the compounds described in the following references: U.S. Pat. No. 6,803,362; US Patent Application Publication Nos. 2004/0105856, 2004/0127395, 2004/0132715, 2004/0048878, 2004/0058934, 2005/0070527, 2005/0070550, and 2005/0261309; and U.S. Provisional Application Nos. ______, filed Mar. 31, 2006 (Attorney Docket No. PRD2650USPSP), ______, filed Mar. 31, 2006 (Attorney Docket No. PRD2651 USPSP), and ______, filed Apr. 7, 2006 (Attorney Docket No. PRD2646USPSP), the disclosures of which are incorporated by reference herein. In one preferred embodiment, the H4R antagonist is a compound as defined in any of the claims of US Patent Application Publication No. 2004/0132715. In a further preferred embodiment, the H4R antagonist is a compound as defined in any of the claims of US Patent Application Publication No. 2004/0048878. In an additional preferred embodiment, the H4R antagonist is a compound as defined in any of the claims of US Patent Application Publication No. 2004/0058934. In another preferred embodiment, the H4R antagonist is a compound as defined in any of the claims of US Patent Application Publication No. 2005/0070527. In a further preferred embodiment, the H4R antagonist is a compound as defined in any of the claims of US Patent Application Publication No. 2005/0070550. In yet another preferred embodiment, the H4R antagonist is a compound as defined in any of the claims of US Patent Application Publication No 2005/0261309. In a further preferred embodiment, the H4R antagonist is a compound as defined in any of the claims of US Patent Application Publication No. 2005/0085487. In another preferred embodiment, the H4R antagonist is a compound as defined in any of the claims of the US provisional application corresponding to Attorney Docket No. PRD2650USPSP. In an alternative preferred embodiment, the H4R antagonist is a compound as defined in any of the claims of the US provisional application corresponding to Attorney Docket No. PRD2651 USPSP. In yet another preferred embodiment, the H4R antagonist is a compound as defined in any of the claims of the US provisional application corresponding to Attorney Docket No. PRD2646USPSP. Particularly preferred H4R antagonists are selected from the compounds listed in Table 1 below and their pharmaceutically acceptable salts:

TABLE 1
(1H-Benzoimidazol-2-yl)-(4-methyl-piperazin-1-yl)-methanone;
(1H-Benzoimidazol-2-yl)-(4-ethyl-piperazin-1-yl)-methanone;
(1H-Benzoimidazol-2-yl)-(3-methyl-piperazin-1-yl)-methanone;
(1H-Benzoimidazol-2-yl)-(4-methyl-[1,4]diazepan-1-yl)-methanone;
1H-Benzoimidazole-2-carboxylic acid (8-methyl-8-aza-bicyclo[3.2.1]oct-3-yl)-amide;
(5-Chloro-1H-benzoimidazol-2-yl)-(4-methyl-piperazin-1-yl)-methanone;
(5-Chloro-1H-benzoimidazol-2-yl)-piperazin-1-yl-methanone;
(5-Chloro-1H-benzoimidazol-2-yl)-(3-methyl-piperazin-1-yl)-methanone;
(5,6-Difluoro-1H-benzoimidazol-2-yl)-(4-methyl-piperazin-1-yl)-methanone;
(5,6-Difluoro-1H-benzoimidazol-2-yl)-(4-ethyl-piperazin-1-yl)-methanone;
(5,6-Difluoro-1H-benzoimidazol-2-yl)-(3-methyl-piperazin-1-yl)-methanone;
(5,6-Difluoro-1H-benzoimidazol-2-yl)-(4-methyl-[1,4]diazepan-1-yl)-
methanone;
5,6-Difluoro-1H-benzoimidazole-2-carboxylic acid (8-methyl-8-aza-
bicyclo[3.2.1]oct-3-yl)-amide;
(6-Chloro-5-fluoro-1H-benzoimidazol-2-yl)-(4-methyl-piperazin-1-yl)-methanone;
(6-Chloro-5-fluoro-1H-benzoimidazol-2-yl)-piperazin-1-yl-methanone;
(6-Chloro-5-fluoro-1H-benzoimidazol-2-yl)-(4-methyl-[1,4]diazepan-1-yl)-
methanone;
6-Chloro-5-fluoro-1H-benzoimidazole-2-carboxylic acid (8-methyl-8-aza-
bicyclo[3.2.1]oct-3-yl)-amide;
(5-Chloro-6-methyl-1H-benzoimidazol-2-yl)-(4-methyl-piperazin-1-yl)-
methanone;
(5-Chloro-6-methyl-1H-benzoimidazol-2-yl)-piperazin-1-yl-methanone;
(4-Methyl-1H-benzoimidazol-2-yl)-(3-methyl-piperazin-1-yl)-methanone;
(4-Ethyl-piperazin-1-yl)-(4-methyl-1H-benzoimidazol-2-yl)-methanone;
(4-Methyl-1H-benzoimidazol-2-yl)-(4-methyl-piperazin-1-yl)-methanone;
(4-Methyl-1H-benzoimidazol-2-yl)-piperazin-1-yl-methanone;
4-Methyl-1H-benzoimidazole-2-carboxylic acid (8-methyl-8-aza-
bicyclo[3.2.1]oct-3-yl)-amide;
5-Methyl-1H-benzoimidazole-2-carboxylic acid (8-methyl-8-aza-
bicyclo[3.2.1]oct-3-yl)-amide;
(5-Methyl-1H-benzoimidazol-2-yl)-(4-methyl-piperazin-1-yl)-methanone;
(4-Methyl-piperazin-1-yl)-(5-trifluoromethyl-1H-benzoimidazol-2-yl)-
methanone;
Piperazin-1-yl-(5-trifluoromethyl-1H-benzoimidazol-2-yl)-methanone;
(5-Fluoro-1H-benzoimidazol-2-yl)-(4-methyl-piperazin-1-yl)-methanone;
(4-Ethyl-piperazin-1-yl)-(5-fluoro-1H-benzoimidazol-2-yl)-methanone;
(5-Fluoro-1H-benzoimidazol-2-yl)-piperazin-1-yl-methanone;
(5-Fluoro-1H-benzoimidazol-2-yl)-(3-methyl-piperazin-1-yl)-methanone;
5-Fluoro-1H-benzoimidazole-2-carboxylic acid (8-methyl-8-aza-
bicyclo[3.2.1]oct-3-yl)-amide;
(3H-Imidazo[4,5-b]pyridin-2-yl)-(4-methyl-piperazin-1-yl)-methanone;
Benzooxazol-2-yl-(4-methyl-piperazin-1-yl)-methanone;
(7-Methyl-benzooxazol-2-yl)-(4-methyl-piperazin-1-yl)-methanone;
(5-Methyl-benzooxazol-2-yl)-(4-methyl-piperazin-1-yl)-methanone;
(4-Methyl-benzooxazol-2-yl)-(4-methyl-piperazin-1-yl)-methanone;
Benzothiazol-2-yl-(4-methyl-piperazin-1-yl)-methanone;
(5-Benzoyl-1H-benzoimidazol-2-yl)-(4-methyl-piperazin-1-yl)-methanone;
(4-Chloro-1H-benzoimidazol-2-yl)-(4-methyl-piperazin-1-yl)-methanone;
(4-Methyl-piperazin-1-yl)-(4-nitro-1H-benzoimidazol-2-yl)-methanone;
(4-Amino-1H-benzoimidazol-2-yl)-(4-methyl-piperazin-1-yl)-methanone;
(4-Isopropylamino-1H-benzoimidazol-2-yl)-(4-methyl-piperazin-1-yl)-
methanone;
C-(5-Chloro-1H-benzoimidazol-2-yl)-C-(4-methyl-piperazin-1-yl)-
methyleneamine;
(4,6-Difluoro-1H-benzoimidazol-2-yl)-(4-methyl-piperazin-1-yl)-methanone;
(4-Methyl-piperazin-1-yl)-(5-nitro-1H-benzoimidazol-2-yl)-methanone;
(5-Fluoro-4-methyl-1H-benzoimidazol-2-yl)-(4-methyl-piperazin-1-yl)-
methanone;
(5-Bromo-1H-benzoimidazol-2-yl)-(4-methyl-piperazin-1-yl)-methanone;
(5,6-Dichloro-1H-benzoimidazol-2-yl)-(4-methyl-piperazin-1-yl)-methanone;
(4,5-Dimethyl-1H-benzoimidazol-2-yl)-(4-methyl-piperazin-1-yl)-methanone;
(5,6-Dimethyl-1H-benzoimidazol-2-yl)-(4-methyl-piperazin-1-yl)-methanone;
(5-Methoxy-1H-benzoimidazol-2-yl)-(4-methyl-piperazin-1-yl)-methanone;
(5-Chloro-benzooxazol-2-yl)-(4-methyl-piperazin-1-yl)-methanone;
(5-Fluoro-benzooxazol-2-yl)-(4-methyl-piperazin-1-yl)-methanone;
(6-Fluoro-benzooxazol-2-yl)-(4-methyl-piperazin-1-yl)-methanone;
(5,7-Difluoro-benzooxazol-2-yl)-(4-methyl-piperazin-1-yl)-methanone;
(4-Methyl-piperazin-1-yl)-(5-trifluoromethoxy-benzooxazol-2-yl)-methanone;
(5-Chloro-benzothiazol-2-yl)-(4-methyl-piperazin-1-yl)-methanone;
(4-Methyl-piperazin-1-yl)-(5-trifluoromethyl-benzothiazol-2-yl)-methanone;
(4-Methyl-piperazin-1-yl)-(6H-thieno[2,3-b]pyrrol-5-yl)-methanone;
(Hexahydro-pyrrolo[1,2-a]pyrazin-2-yl)-(6H-thieno[2,3-b]pyrrol-5-yl)-
methanone;
(2-Chloro-6H-thieno[2,3-b]pyrrol-5-yl)-(4-methyl-piperazin-1-yl)-methanone;
(2-Chloro-6H-thieno[2,3-b]pyrrol-5-yl)-(hexahydro-pyrrolo[1,2-a]pyrazin-2-
yl)-methanone;
(2-Chloro-6H-thieno[2,3-b]pyrrol-5-yl)-piperazin-1-yl-methanone;
(4H-Furo[3,2-b]pyrrol-5-yl)-(4-methyl-piperazin-1-yl)-methanone;
(4-Methyl-piperazin-1-yl)-(4H-thieno[3,2-b]pyrrol-5-yl)-methanone;
Piperazin-1-yl-(4H-thieno[3,2-b]pyrrol-5-yl)-methanone;
(3-Methyl-piperazin-1-yl)-(4H-thieno[3,2-b]pyrrol-5-yl)-methanone;
(2-Chloro-4H-thieno[3,2-b]pyrrol-5-yl)-(4-methyl-piperazin-1-yl)-methanone;
(2-Chloro-4H-thieno[3,2-b]pyrrol-5-yl)-(hexahydro-pyrrolo[1,2-a]pyrazin-2-
yl)-methanone;
(3-Bromo-4H-thieno[3,2-b]pyrrol-5-yl)-(4-methyl-piperazin-1-yl)-methanone;
4-Methyl-piperazin-1-yl)-(3-methyl-4H-thieno[3,2-b]pyrrol-5-yl)-methanone;
(2-Methyl-4H-furo[3,2-b]pyrrol-5-yl)-(4-methyl-piperazin-1-yl)-methanone;
(2,3-Dimethyl-4H-furo[3,2-b]pyrrol-5-yl)-(4-methyl-piperazin-1-yl)-
methanone;
(2,3-Dimethyl-4H-thieno[3,2-b]pyrrol-5-yl)-(4-methyl-piperazin-1-yl)-
methanone;
(2,3-Dichloro-6H-thieno[2,3-b]pyrrol-5-yl)-(4-methyl-piperazin-1-yl)-
methanone;
(2-Methyl-4H-furo[3,2-b]pyrrol-5-yl)-piperazin-1-yl-
methanone;
(3-Bromo-4H-thieno[3,2-b]pyrrol-5-yl)-piperazin-1-yl-methanone;
(3-Bromo-4H-thieno[3,2-b]pyrrol-5-yl)-(3-methyl-piperazin-1-yl)-
methanone;
(3-Methyl-4H-thieno[3,2-b]pyrrol-5-yl)-piperazin-1-yl-methanone;
(3-Methyl-piperazin-1-yl)-(3-methyl-4H-thieno[3,2-b]pyrrol-5-yl)-methanone;
(2-Chloro-3-methyl-4H-thieno[3,2-b]pyrrol-5-yl)-(4-methyl-piperazin-1-yl)-
methanone;
(2-Chloro-3-methyl-4H-thieno[3,2-b]pyrrol-5-yl)-piperazin-1-yl-methanone;
(2,3-Dichloro-4H-thieno[3,2-b]pyrrol-5-yl)-(4-methyl-piperazin-1-yl)-methanone;
(2,3-Dimethyl-6H-thieno[2,3-b]pyrrol-5-yl)-(4-methyl-piperazin-1-yl)-methanone;
(2-Chloro-3-methyl-6H-thieno[2,3-b]pyrrol-5-yl)-(4-methyl-piperazin-1-yl)-
methanone;
(3-Chloro-2-methyl-6H-thieno[2,3-b]pyrrol-5-yl)-(4-methyl-piperazin-1-yl)-
methanone;
(2-Bromo-6H-thieno[2,3-b]pyrrol-5-yl)-(4-methyl-piperazin-1-yl)-methanone;
(3-Bromo-6H-thieno[2,3-b]pyrrol-5-yl)-(4-methyl-piperazin-1-yl)-methanone;
(4-Methyl-piperazin-1-yl)-(2-phenyl-6H-thieno[2,3-b]pyrrol-5-yl)-methanone;
[2-(4-Chloro-phenyl)-6H-thieno[2,3-b]pyrrol-5-yl]-(4-methyl-piperazin-1-yl)-
methanone;
(3-Bromo-4H-thieno[3,2-b]pyrrol-5-yl)-(3,4-dimethyl-piperazin-1-yl)-methanone;
(3,4-Dimethyl-piperazin-1-yl)-(3-methyl-4H-thieno[3,2-b]pyrrol-5-yl)-methanone;
(2-Bromo-3-methyl-4H-thieno[3,2-b]pyrrol-5-yl)-(4-methyl-piperazin-1-yl)-
methanone;
(3-Bromo-2-chloro-4H-thieno[3,2-b]pyrrol-5-yl)-(4-methyl-piperazin-1-yl)-
methanone;
(2,3-Dichloro-4H-thieno[3,2-b]pyrrol-5-yl)-(3-methyl-piperazin-1-yl)-methanone;
(4-Methyl-piperazin-1-yl)-(2-phenyl-4H-thieno[3,2-b]pyrrol-5-yl)-methanone;
(4-Methyl-piperazin-1-yl)-[2-(4-trifluoromethyl-phenyl)-4H-thieno[3,2-b]-
pyrrol-5-yl]-methanone;
8-Methyl-3-(4-methyl-piperazin-1-yl)-1H-quinoxalin-2-one;
8-Methyl-3-piperazin-1-yl-1H-quinoxalin-2-one;
8-Nitro-3-piperazin-1-yl-1H-quinoxalin-2-one;
7,8-Difluoro-3-piperazin-1-yl-1H-quinoxalin-2-one;
8-Methyl-3-(3-methyl-piperazin-1-yl)-1H-quinoxalin-2-one;
3-(3-Methyl-piperazin-1-yl)-1H-quinoxalin-2-one;
3-[4-(2-Hydroxy-ethyl)-piperazin-1-yl]-8-methyl-1H-quinoxalin-2-one;
6-Chloro-3-(4-methyl-piperazin-1-yl)-1H-quinoxalin-2-one;
7-Chloro-3-(4-methyl-piperazin-1-yl)-1H-quinoxalin-2-one;
3-(4-Methyl-piperazin-1-yl)-6-trifluoromethyl-1H-quinoxalin-2-one;
3-(4-Methyl-piperazin-1-yl)-7-trifluoromethyl-1H-quinoxalin-2-one;
6,7-Dichloro-3-(4-methyl-piperazin-1-yl)-1H-quinoxalin-2-one;
6,7-Dichloro-3-piperazin-1-yl-1H-quinoxalin-2-one;
6,7-Dichloro-3-(4-methyl-[1,4]diazepan-1-yl)-1H-quinoxalin-2-one;
6,7-Difluoro-3-(4-methyl-piperazin-1-yl)-1H-quinoxalin-2-one;
7-Chloro-6-methyl-3-(4-methyl-piperazin-1-yl)-1H-quinoxalin-2-one;
6-Chloro-7-methyl-3-(4-methyl-piperazin-1-yl)-1H-quinoxalin-2-one;
6-Fluoro-3-(4-methyl-piperazin-1-yl)-1H-quinoxalin-2-one;
7,8-Difluoro-3-(4-methyl-piperazin-1-yl)-1H-quinoxalin-2-one;
8-Chloro-3-(4-methyl-piperazin-1-yl)-6-trifluoromethyl-1H-quinoxalin-2-one;
3-Piperazin-1-yl-6-trifluoromethyl-1H-quinoxalin-2-one;
3-Piperazin-1-yl-7-trifluoromethyl-1H-quinoxalin-2-one;
6-Chloro-7-fluoro-3-(4-methyl-piperazin-1-yl)-1H-quinoxalin-2-one;
7-Chloro-6-fluoro-3-(4-methyl-piperazin-1-yl)-1H-quinoxalin-2-one;
7-Chloro-3-piperazin-1-yl-1H-quinoxalin-2-one;
6-Chloro-3-piperazin-1-yl-1H-quinoxalin-2-one;
6-Chloro-3-(3-methyl-piperazin-1-yl)-1H-quinoxalin-2-one;
7-Chloro-3-(3-methyl-piperazin-1-yl)-1H-quinoxalin-2-one;
3-(3-Methyl-piperazin-1-yl)-6-trifluoromethyl-1H-quinoxatin-2-one;
3-(3-Methyl-piperazin-1-yl)-7-trifluoromethyl-1H-quinoxalin-2-one;
2-{2-Chloro-4-[3-(4-methyl-piperazin-1-yl)-propoxy]-phenyl}-4,5-
dimethyl-1H-benzoimidazole;
2-{2-Chloro-4-[3-(1-methyl-piperidin-4-yl)-propoxy]-phenyl}-4-methyl-1H-
benzoimidazole;
2-{2-Chloro-4-[3-(4-methyl-piperazin-1-yl)-propoxy]-phenyl}-5-
trifluoromethoxy-1H-benzoimidazole;
5-tert-Butyl-2-{3-chloro-4-[3-(4-methyl-[1,4]diazepan-1-yl)-propoxy]-
phenyl}-1H-benzoimidazole;
5-tert-Butyl-2-{3-chloro-4-[3-(4-methyl-piperazin-1-yl)-propoxy]-phenyl}-
1H-benzoimidazole;
4,5-Dimethyl-2-{3-methyl-4-[3-(4-methyl-piperazin-1-yl)-propoxy]-
phenyl}-1H-benzoimidazole;
5-tert-Butyl-2-{3-[4-(4-methyl-piperazin-1-yl)-butoxy]-phenyl}-1H-
benzoimidazole;
5-tert-Butyl-2-{3-[4-(4-methyl-[1,4]diazepan-1-yl)-butoxy]-phenyl}-1H-
benzoimidazole;
(1-{3-[4-(5-tert-Butyl-1H-benzoimidazol-2-yl)-2-chloro-phenoxy]-propyl}-
pyrrolidin-3-yl)-dimethylamine;
5-Chloro-2-{3-chloro-4-[3-(4-methyl-[1,4]diazepan-1-yl)-propoxy]-
phenyl}-6-methyl-1H-benzoimidazole;
2-{3-Fluoro-4-[3-(4-methyl-piperazin-1-yl)-propoxy]-phenyl}-4-methyl-
1H-benzoimidazole;
5-Methyl-2-{4-[3-(4-methyl-piperazin-1-yl)-propoxy]-naphthalen-1-yl}-
1H-benzoimidazole;
4-[3-(5-tert-Butyl-1H-benzoimidazol-2-yl)-phenoxy]-1-(4-methyl-
piperazin-1-yl)-butan-1-one;
5-Chloro-2-[3-chloro-4-(3-piperazin-1-yl-propoxy)-phenyl]-6-fluoro-1H-
benzoimidazole;
5-tert-Butyl-2-{3-methyl-4-[3-(4-methyl-piperazin-1-yl)-propoxy]-phenyl}-
1H-benzoimidazole;
2-{2-Chloro-4-[3-(1-methyl-piperidin-4-yl)-propoxy]-phenyl}-4,6-
dimethyl-1H-benzoimidazole;
2-{2-Chloro-4-[2-methyl-3-(4-methyl-piperazin-1-yl)-propoxy]-phenyl}-4-
methyl-1H-benzoimidazole;
5-Chloro-2-{3-chloro-4-[3-(4-methyl-piperazin-1-yl)-propoxy]-phenyl}-6-
methyl-1H-benzoimidazole;
6-Chloro-2-{2-chloro-4-[3-(4-methyl-piperazin-1-yl)-propoxy]-phenyl}-4-
methyl-1H-benzoimidazole;
5-tert-Butyl-2-{3-chloro-4-[3-(4-methyl-piperazin-1-yl)-propoxy]-phenyl}-
1H-benzoimidazole;
5-Chloro-2-{3-fluoro-4-[3-(4-methyl-piperazin-1-yl)-propoxy]-phenyl}-1H-
benzoimidazole;
2-{2-Chloro-4-[3-(4-methyl-[1,4]diazepan-1-yl)-propoxy]-phenyl}-4,6-
dimethyl-1H-benzoimidazole;
5-Chloro-6-methyl-2-{3-[4-(4-methyl-piperazin-1-yl)-butoxy]-phenyl}-1H-
benzoimidazole;
5-Chloro-6-fluoro-2-{3-fluoro-4-[3-(4-methyl-piperazin-1-yl)-propoxy]-
phenyl}-1H-benzoimidazole;
2-{3-Fluoro-4-[3-(4-methyl-piperazin-1-yl)-propoxy]-phenyl}-5-methyl-
1H-benzoimidazole;
5,6-Difluoro-2-{3-fluoro-4-[3-(4-methyl-piperazin-1-yl)-propoxy]-phenyl}-
1H-benzoimidazole;
2-{3-Fluoro-4-[3-(4-methyl-piperazin-1-yl)-propoxy]-phenyl}-1H-
benzoimidazole;
2-{2-Chloro-4-[3-(4-methyl-[1,4]diazepan-1-yl)-propoxy]-phenyl}-4,5-
dimethyl-1H-benzoimidazole;
5,6-Dimethyl-2-{3-[4-(4-methyl-piperazin-1-yl)-butoxy]-phenyl}-1H-
benzoimidazole;
2-{2-Chloro-4-[3-(4-methyl-piperazin-1-yl)-propoxy]-phenyl}-4,6-
dimethyl-1H-benzoimidazole;
2-{2-Chloro-4-[3-(4-methyl-[1,4]diazepan-1-yl)-propoxy]-phenyl}-4-
methyl-1H-benzoimidazole;
5-tert-Butyl-2-{2-chloro-4-[3-(4-methyl-piperazin-1-yl)-propoxy]-phenyl}-
1H-benzoimidazole;
2-{3-Methoxy-4-[3-(4-methyl-piperazin-1-yl)-propoxy]-phenyl}-5-trifluoromethyl-1H-
benzoimidazole;
5-Chloro-2-{3-chloro-4-[3-(4-methyl-piperazin-1-yl)-propoxy]-phenyl}-6-fluoro-1H-
benzoimidazole;
5,6-Dichloro-2-{2-chloro-4-[3-(4-methyl-piperazin-1-yl)-propoxy]-
phenyl}-1H-benzoimidazole;
5-Chloro-2-{2-chloro-4-[3-(4-methyl-piperazin-1-yl)-propoxy]-phenyl}-
1H-benzoimidazole;
5-Chloro-2-{2-chloro-4-[3-(4-methyl-piperazin-1-yl)-propoxy]-phenyl}-
6-fluoro-1H-benzoimidazole;
5-Chloro-2-{3-methyl-4-[3-(4-methyl-piperazin-1-yl)-propoxy]-
phenyl}-1H-benzoimidazole;
2-{3-Chloro-4-[3-(4-methyl-piperazin-1-yl)-propoxy]-phenyl}-5-
methyl-1H-benzoimidazole;
5,6-Dichloro-2-{3-chloro-4-[3-(4-methyl-piperazin-1-yl)-propoxy]-
phenyl}-1H-benzoimidazole;
5-Chloro-6-methyl-2-{3-methyl-4-[3-(4-methyl-piperazin-1-yl)-
propoxy]-phenyl}-1H-benzoimidazole;
2-{2-Chloro-4-[3-(4-methyl-piperazin-1-yl)-propoxy]-phenyl}-5-
methyl-1H-benzoimidazole;
5-Chloro-2-{3-chloro-4-[3-(4-methyl-piperazin-1-yl)-propoxy]-phenyl}-
1H-benzoimidazole;
2-{3-Chloro-4-[3-(4-methyl-piperazin-1-yl)-propoxy]-phenyl}-5-
trifluoromethyl-1H-benzoimidazole;
5-Chloro-6-fluoro-2-{3-methyl-4-[3-(4-methyl-piperazin-1-yl)-
propoxy]-phenyl}-1H-benzoimidazole;
5-Methyl-2-{3-methyl-4-[3-(4-methyl-piperazin-1-yl)-propoxy]-
phenyl}-1H-benzoimidazole;
2-{3-Chloro-4-[3-(4-methyl-piperazin-1-yl)-propoxy]-phenyl}-1H-
benzoimidazole;
2-{3-Methyl-4-[3-(4-methyl-piperazin-1-yl)-propoxy]-phenyl}-1H-
benzoimidazole;
2-{2-Chloro-4-[3-(4-methyl-piperazin-1-yl)-propoxy]-phenyl}-1H-
benzoimidazole;
5-Chloro-6-fluoro-2-{3-methoxy-4-[3-(4-methyl-piperazin-1-yl)-
propoxy]-phenyl}-1H-benzoimidazole;
2-{3-Chloro-4-[3-(4-methyl-piperazin-1-yl)-propoxy]-phenyl}-5-
methoxy-1H-benzoimidazole;
5-tert-Butyl-2-{3,5-dibromo-4-[3-(4-methyl-piperazin-1-yl)-propoxy]-
phenyl}-1H-benzoimidazole;
2-{2-Methoxy-4-[3-(4-methyl-piperazin-1-yl)-propoxy]-phenyl}-5-
trifluoromethyl-1H-benzoimidazole;
2-{2-Chloro-4-[3-(4-methyl-piperazin-1-yl)-propoxy]-phenyl}-5-
trifluoromethyl-1H-benzoimidazole;
2-{3-[3-(4-Methyl-piperazin-1-yl)-propoxy]-phenyl}-1H-
benzoimidazole;
(2-{3-[4-(4-Methyl-piperazin-1-yl)-butoxy]-phenyl}-1H-benzoimidazol-
5-yl)-phenyl-methanone;
6-Chloro-2-{2-chloro-4-[3-(1-methyl-piperidin-4-yl)-propoxy]-phenyl}-
4-methyl-1H-benzoimidazole;
5-tert-Butyl-2-{3-chloro-4-[3-(1-methyl-piperidin-4-yl)-propoxy]-
phenyl}-1H-benzoimidazole;
2-{2-Chloro-4-[3-(1-methyl-piperidin-4-yl)-propoxy]-phenyl}-4,5-
dimethyl-1H-benzoimidazole;
5-Chloro-6-methyl-2-{4-[3-(1-methyl-piperidin-4-yl)-propoxy]-phenyl}-
1H-benzoimidazole;
5-Chloro-2-{4-[3-(1-methyl-piperidin-4-yl)-propoxy]-phenyl}-1H-
benzoimidazole;
5-Chloro-6-fluoro-2-{4-[3-(1-methyl-piperidin-4-yl)-propoxy]-phenyl}-
1H-benzoimidazole;
5-tert-Butyl-2-{4-[3-(1-methyl-piperidin-4-yl)-propoxy]-phenyl}-1H-
benzoimidazole;
5-Methyl-2-{4-[3-(1-methyl-piperidin-4-yl)-propoxy]-phenyl}-1H-
benzoimidazole;
2-{4-[3-(1-Methyl-piperidin-4-yl)-propoxy]-phenyl}-1H-
benzoimidazole;
6-Chloro-2-{2-fluoro-4-[3-(1-methyl-piperidin-4-yl)-propoxy]-phenyl}-
4-methyl-1H-benzoimidazole;
5-Fluoro-2-{2-methyl-4-[3-(1-methyl-piperidin-4-yl)-propoxy]-phenyl}-
1H-benzoimidazole;
4-Chloro-2-{2-methyl-4-[3-(1-methyl-piperidin-4-yl)-propoxy]-phenyl}-
1H-benzoimidazole;
6-Chloro-4-methyl-2-{2-methyl-4-[3-(1-methyl-piperidin-4-yl)-
propoxy]-phenyl}-1H-benzoimidazole;
5-Chloro-2-{2-chloro-4-[3-(1-methyl-piperidin-4-yl)-propoxy]-phenyl}-
6-fluoro-1H-benzoimidazole;
2-{2-Chloro-4-[3-(1-methyl-piperidin-4-yl)-propoxy]-phenyl}-3H-
naphtho[1,2-d]imidazole;
4,6-Dimethyl-2-{2-methyl-4-[3-(4-methyl-piperazin-1-yl)-propoxy]-
phenyl}-1H-benzoimidazole;
2-{2-Chloro-4-[3-(4-methyl-piperazin-1-yl)-propoxy]-phenyl}-4-
methyl-1H-benzoimidazole;
2-{2-Chloro-4-[3-(4-methyl-piperazin-1-yl)-propoxy]-phenyl}-5-fluoro-
4-methyl-1H-benzoimidazole;
2-{2-Chloro-4-[3-(4-methyl-piperazin-1-yl)-propoxy]-phenyl}-3H-
naphtho[1,2-d]imidazole;
6-{2-Chloro-4-[3-(4-methyl-piperazin-1-yl)-propoxy]-phenyl}-5H-
[1,3]dioxolo[4′,5′:4,5]benzo[1,2-d]imidazole;
6-Chloro-2-{2-chloro-4-[3-(4-methyl-[1,4]diazepan-1-yl)-propoxy]-
phenyl}-4-methyl-1H-benzoimidazole;
2-{3-Chloro-4-[3-(4-methyl-[1,4]diazepan-1-yl)-propoxy]-phenyl}-4-
methyl-1H-benzoimidazole;
4,6-Dimethyl-2-{3-[4-(4-methyl-[1,4]diazepan-1-yl)-butoxy]-phenyl}-
1H-benzoimidazole;
5-Chloro-2-{4-[3-(4-methyl-piperazin-1-yl)-propoxy]-phenyl}-1H-
benzoimidazole;
2-{4-[3-(4-Methyl-piperazin-1-yl)-propoxy]-phenyl}-1H-
benzoimidazole;
{2-(6-Chloro-4-methyl-1H-benzoimidazol-2-yl)-5-[3-(1-methyl-
piperidin-4-yl)-propoxy]-benzyl}-dimethyl-amine;
{2-(5-Fluoro-4-methyl-1H-benzoimidazol-2-yl)-5-[3-(1-methyl-
piperidin-4-yl)-propoxy]-benzyl}-dimethyl-amine;
4-{3-[4-(6-Chloro-4-methyl-1H-benzoimidazol-2-yl)-3-methyl-
phenoxy]-propyl}-[1,4]diazepan-5-one;
4-{3-[4-(5-tert-Butyl-1H-benzoimidazol-2-yl)-3-methyl-phenoxy]-
propyl}-1-methyl-[1,4]diazepan-5-one;
5-tert-Butyl-2-{2-methyl-4-[3-(2-methyl-piperazin-1-yl)-propoxy]-
phenyl}-1H-benzoimidazole;
5-tert-Butyl-2-{2-methyl-4-[3-(2-methyl-piperazin-1-yl)-propoxy]-
phenyl}-1H-benzoimidazole;
6-Chloro-4-methyl-2-[2-methyl-4-(3-piperidin-4-yl-propoxy)-phenyl]-
1H-benzoimidazole;
5-Fluoro-4-methyl-2-[2-methyl-4-(3-piperidin-4-yl-propoxy)-phenyl]-
1H-benzoimidazole;
6-Chloro-2-{4-[3-(1-ethyl-piperidin-4-yl)-propoxy]-2-methyl-phenyl}-4-
methyl-1H-benzoimidazole;
{2-[3-Chloro-4-(4-methyl-1H-benzoimidazol-2-yl)-phenoxy]-ethyl}-
methyl-(1-methyl-piperidin-4-yl)-amine;
6-Chloro-4-methyl-2-{2-methyl-4-[2-(1-methyl-piperidin-4-yloxy)-
ethoxy]-phenyl}-1H-benzoimidazole;
6-Chloro-4-methyl-2-{2-methyl-4-[3-(1-methyl-1,2,3,6-tetrahydro-
pyridin-4-yl)-propoxy]-phenyl}-1H-benzoimidazole;
5-Fluoro-4-methyl-2-{2-methyl-4-[3-(1-methyl-1,2,3,6-tetrahydro-
pyridin-4-yl)-propoxy]-phenyl}-1H-benzoimidazole;
6-Fluoro-7-methyl-2-{3-[4-(1-methyl-piperidin-4-yl)-butoxy]-phenyl}-
1H-benzoimidazole;
7-Methyl-2-{3-[4-(1-methyl-piperidin-4-yl)-butoxy]-phenyl}-1H-
benzoimidazole;
6,7-Dimethyl-2-{3-[4-(1-methyl-piperidin-4-yl)-butoxy]-phenyl}-1H-
benzoimidazole;
(5-Chloro-1H-indol-2-yl)-(4-methyl-piperazin-1-yl)-methanone;
(5-Fluoro-1H-indol-2-yl)-(4-methyl-piperazin-1-yl)-methanone;
(5-Bromo-1H-indol-2-yl)-(4-methyl-piperazin-1-yl)-methanone;
(1H-Indol-2-yl)-(4-methyl-piperazin-1-yl)-methanone;
(5-Benzyloxy-1H-indol-2-yl)-(4-methyl-piperazin-1-yl)-methanone;
(5-Methyl-1H-indol-2-yl)-(4-methyl-piperazin-1-yl)-methanone;
(5,6-Dimethoxy-1H-indol-2-yl)-(4-methyl-piperazin-1-yl)-methanone;
(4-Methyl-piperazin-1-yl)-(7-nitro-1H-indol-2-yl)-methanone;
(4-Methyl-piperazin-1-yl)-(5-nitro-3-phenyl-1H-indol-2-yl)-methanone;
(4-Methyl-piperazin-1-yl)-(5-trifluoromethoxy-1H-indol-2-yl)-methanone;
(6-Chloro-1H-indol-2-yl)-(4-methyl-piperazin-1-yl)-methanone;
(5,7-Difluoro-1H-indol-2-yl)-(4-methyl-piperazin-1-yl)-methanone;
(6-Fluoro-1H-indol-2-yl)-(4-methyl-piperazin-1-yl)-methanone;
(4,6-Dichloro-1H-indol-2-yl)-(4-methyl-piperazin-1-yl)-methanone;
(1H-Indol-2-yl)-(4-octyl-piperazin-1-yl)-methanone;
(4-Ethyl-piperazin-1-yl)-(1H-indol-2-yl)-methanone;
(1H-Indol-2-yl)-(4-isopropyl-piperazin-1-yl)-methanone;
[4-(3-Dimethylamino-propyl)-piperazin-1-yl]-(1H-indol-2-yl)-methanone;
(4-Butyl-piperazin-1-yl)-(1H-indol-2-yl)-methanone;
(4-Cyclopentyl-piperazin-1-yl)-(1H-indol-2-yl)-methanone;
(1H-Indol-2-yl)-(4-phenethyl-piperazin-1-yl)-methanone;
(1H-Indol-2-yl)-[4-(2-piperidin-1-yl-ethyl)-piperazin-1-yl]-methanone;
[4-(2-Ethoxy-ethyl)-piperazin-1-yl]-(1H-indol-2-yl)-methanone;
(4-sec-Butyl-piperazin-1-yl)-(1H-indol-2-yl)-methanone;
[4-(1-Ethyl-propyl)-piperazin-1-yl]-(1H-indol-2-yl)-methanone;
(1H-Indol-2-yl)-[4-(3-phenyl-propyl)-piperazin-1-yl]-methanone;
(1H-Indol-2-yl)-[4-(1-methyl-piperidin-4-yl)-piperazin-1-yl]-methanone;
[4-(2-Dipropylamino-ethyl)-piperazin-1-yl]-(1H-indol-2-yl)-methanone;
(1H-Indol-2-yl)-[4-(3-phenyl-allyl)-piperazin-1-yl]-methanone;
(1H-Indol-2-yl)-(4-pentyl-piperazin-1-yl)-methanone;
(4-Heptyl-piperazin-1-yl)-(1H-indol-2-yl)-methanone;
[4-(2-Diethylamino-ethyl)-piperazin-1-yl]-(1H-indol-2-yl)-methanone;
(1H-Indol-2-yl)-[4-(4-methoxy-butyl)-piperazin-1-yl]-methanone;
5-Chloro-7-methyl-2-{3-[4-(1-methyl-piperidin-4-yl)-butoxy]-phenyl}-1H-
benzoimidazole;
5,7-Dimethyl-2-{2-methyl-3-[4-(1-methyl-piperidin-4-yl)-butoxy]-phenyl}-1H-
benzoimidazole;
5-Chloro-7-methyl-2-{2-methyl-3-[4-(1-methyl-piperidin-4-yl)-butoxy]-
phenyl}-1H-benzoimidazole;
6-Fluoro-7-methyl-2-{2-methyl-3-[4-(1-methyl-piperidin-4-yl)-butoxy]-
phenyl}-1H-benzoimidazole;
6-Fluoro-7-methyl-2-{3-[3-(1-methyl-piperidin-4-yloxy)-propoxy]-phenyl}-1H-
benzoimidazole;
{2-(5-Fluoro-4-methyl-1H-benzoimidazol-2-yl)-5-[3-(1-methyl-piperidin-4-yl)-
propoxy]-phenyl}-methanol;
6-Chloro-4-methyl-2-{6-[3-(4-methyl-piperazin-1-yl)-propoxy]-pyridin-3-yl}-1H-
benzoimidazole;
4-Methyl-2-{6-[3-(4-methyl-piperazin-1-yl)-propoxy]-pyridin-3-yl}-1H-
benzoimidazole;
5-Fluoro-4-methyl-2-{6-[3-(4-methyl-piperazin-1-yl)-propoxy]-pyridin-3-yl}-1H-
benzoimidazole;
4-Methyl-2-{6-[3-(1-methyl-piperidin-4-yl)-propoxy]-pyridin-3-yl}-1H-
benzoimidazole;
4,5-Dimethyl-2-{6-[3-(1-methyl-piperidin-4-yl)-propoxy]-pyridin-3-yl}-1H-
benzoimidazole;
4-Chloro-2-{6-[3-(1-methyl-piperidin-4-yl)-propoxy]-pyridin-3-yl}-1H-
benzoimidazole;
6-Chloro-4-methyl-2-{4-methyl-6-[3-(1-methyl-piperidin-4-yl)-propoxy]-pyridin-3-
yl}-1H-benzoimidazole;
4-Methyl-2-{4-methyl-6-[3-(1-methyl-piperidin-4-yl)-propoxy]-pyridin-3-yl}-1H-
benzoimidazole;
5-Fluoro-4-methyl-2-{4-methyl-6-[3-(1-methyl-piperidin-4-yl)-propoxy]-pyridin-3-
yl}-1H-benzoimidazole;
4,5-Dimethyl-2-{4-methyl-6-[3-(1-methyl-piperidin-4-yl)-propoxy]-pyridin-3-yl}-1H-
benzoimidazole;
4,6-Dimethyl-2-{4-methyl-6-[3-(1-methyl-piperidin-4-yl)-propoxy]-pyridin-3-yl}-
1H-benzoimidazole;
4-Chloro-2-{4-methyl-6-[3-(1-methyl-piperidin-4-yl)-propoxy]-pyridin-3-yl}-1H-
benzoimidazole;
2-{4-Chloro-6-[3-(1-methyl-piperidin-4-yl)-propoxy]-pyridin-3-yl}-5-fluoro-4-
methyl-1H-benzoimidazole;
2-{4-Chloro-6-[3-(1-methyl-piperidin-4-yl)-propoxy]-pyridin-3-yl}-4-methyl-1H-
benzoimidazole;
6-Chloro-2-{4-chloro-6-[3-(1-methyl-piperidin-4-yl)-propoxy]-pyridin-3-yl}-4-
methyl-1H-benzoimidazole;
2-{4-Chloro-6-[3-(1-methyl-piperidin-4-yl)-propoxy]-pyridin-3-yl}-4,6-dimethyl-
1H-benzoimidazole;
2-{4-Methoxy-6-[3-(1-methyl-piperidin-4-yl)-propoxy]-pyridin-3-yl}-4-methyl-1H-
benzoimidazole;
5-Fluoro-2-{4-methoxy-6-[3-(1-methyl-piperidin-4-yl)-propoxy]-pyridin-3-yl}-4-
methyl-1H-benzoimidazole;
5-Fluoro-4-methyl-2-{6-[3-(1-methyl-1,2,3,6-tetrahydro-pyridin-4-yl)-
propoxy]-pyridin-3-yl}-1H-benzoimidazole;
4-Methyl-2-{6-[3-(1-methyl-1,2,3,6-tetrahydro-pyridin-4-yl)-propoxy]-
pyridin-3-yl}-1H-benzoimidazole;
6-Chloro-4-methyl-2-{6-[3-(1-methyl-1,2,3,6-tetrahydro-pyridin-4-yl)-
propoxy]-pyridin-3-yl}-1H-benzoimidazole;
4,5-Dimethyl-2-{6-[3-(1-methyl-1,2,3,6-tetrahydro-pyridin-4-yl)-propoxy]-
pyridin-3-yl}-1H-benzoimidazole;
4,6-Dimethyl-2-{6-[3-(1-methyl-1,2,3,6-tetrahydro-pyridin-4-yl)-propoxy]-
pyridin-3-yl}-1H-benzoimidazole;
5-Chloro-4-methyl-2-{6-[3-(1-methyl-1,2,3,6-tetrahydro-pyridin-4-yl)-
propoxy]-pyridin-3-yl}-1H-benzoimidazole;
5-Fluoro-4-methyl-2-{6-[3-(1-methyl-piperidin-4-yl)-propoxy]-4-pyrrolidin-1-
ylmethyl-pyridin-3-yl}-1H-benzoimidazole;
2-{5-Bromo-2-[4-(1-methyl-piperidin-4-yl)-butoxy]-pyridin-4-yl}-4-methyl-
1H-benzoimidazole;
2-{5-Bromo-2-[4-(1-methyl-piperidin-4-yl)-butoxy]-pyridin-4-yl}-5-fluoro-4-
methyl-1H-benzoimidazole;
2-{5-Bromo-2-[4-(1-methyl-piperidin-4-yl)-butoxy]-pyridin-4-yl}-6-chloro-4-
methyl-1H-benzoimidazole;
2-{5-Bromo-2-[4-(1-methyl-piperidin-4-yl)-butoxy]-pyridin-4-yl}-4,6-
dimethyl-1H-benzoimidazole;
2-{5-Bromo-2-[4-(1-methyl-piperidin-4-yl)-butoxy]-pyridin-4-yl}-4,5-
dimethyl-1H-benzoimidazole;
2-{5-Bromo-2-[4-(1-methyl-piperidin-4-yl)-butoxy]-pyridin-4-yl}-5-chloro-4-
methyl-1H-benzoimidazole;
2-{5-Bromo-2-[4-(1-methyl-piperidin-4-yl)-butoxy]-pyridin-4-yl}-5-tert-butyl-
1H-benzoimidazole;
5-tert-Butyl-2-{2-[4-(1-methyl-piperidin-4-yl)-butoxy]-pyridin-4-yl}-1H-
benzoimidazole;
2-{5-Chloro-2-[4-(1-methyl-piperidin-4-yl)-butoxy]-pyridin-4-yl}-5-fluoro-4-
methyl-1H-benzoimidazole;
2-{5-Chloro-2-[4-(1-methyl-piperidin-4-yl)-butoxy]-pyridin-4-yl}-4,5-
dimethyl-1H-benzoimidazole;
4,6-Dimethyl-2-{2-[4-(4-methyl-piperazin-1-yl)-butoxy]-pyridin-4-yl}-1H-
benzoimidazole;
4-Methyl-2-{2-[4-(4-methyl-piperazin-1-yl)-butoxy]-pyridin-4-yl}-1H-
benzoimidazole;
4,5-Dimethyl-2-{2-[4-(4-methyl-piperazin-1-yl)-butoxy]-pyridin-4-yl}-1H-
benzoimidazole;
5-Fluoro-4-methyl-2-{2-[4-(4-methyl-piperazin-1-yl)-butoxy]-pyridin-4-yl}-
1H-benzoimidazole;
6-Chloro-4-methyl-2-{2-[4-(4-methyl-piperazin-1-yl)-butoxy]-pyridin-4-yl}-
1H-benzoimidazole;
5-Fluoro-4-methyl-2-{2-[4-(4-methyl-[1,4]diazepan-1-yl)-butoxy]-pyridin-4-
yl}-1H-benzoimidazole;
4,5-Dimethyl-2-{2-[4-(4-methyl-[1,4]diazepan-1-yl)-butoxy]-pyridin-4-yl}-
1H-benzoimidazole;
4,6-Dimethyl-2-{2-[4-(1-methyl-piperidin-4-yl)-butoxy]-pyridin-4-yl}-1H-
benzoimidazole;
4-Methyl-2-{2-[4-(1-methyl-piperidin-4-yl)-butoxy]-pyridin-4-yl}-1H-
benzoimidazole;
5-Fluoro-4-methyl-2-{2-[4-(1-methyl-piperidin-4-yl)-butoxy]-pyridin-4-yl}-1H-
benzoimidazole;
4-Chloro-2-{2-[4-(1-methyl-piperidin-4-yl)-butoxy]-pyridin-4-yl}-1H-
benzoimidazole;
4,5-Dimethyl-2-{2-[4-(1-methyl-piperidin-4-yl)-butoxy]-pyridin-4-yl}-1H-
benzoimidazole;
6-Chloro-4-methyl-2-{2-[4-(1-methyl-piperidin-4-yl)-butoxy]-pyridin-4-yl}-1H-
benzoimidazole;
5-Chloro-4-methyl-2-{2-[4-(1-methyl-piperidin-4-yl)-butoxy]-pyridin-4-yl}-1H-
benzoimidazole;
5-tert-Butyl-2-[2-(4-piperidin-4-yl-butoxy)-pyridin-4-yl]-1H-benzoimidazole;
4,6-Dimethyl-2-[2-(4-piperidin-4-yl-butoxy)-pyridin-4-yl]-1H-benzoimidazole;
2-{2-[4-(1-Ethyl-piperidin-4-yl)-butoxy]-pyridin-4-yl}-4,5-dimethyl-1H-
benzoimidazole;
4,6-Dimethyl-2-{3-methyl-2-[4-(1-methyl-piperidin-4-yl)-butoxy]-pyridin-4-yl}-
1H-benzoimidazole;
4-Methyl-2-{3-methyl-2-[4-(1-methyl-piperidin-4-yl)-butoxy]-pyridin-4-yl}-1H-
benzoimidazole;
6-Chloro-4-methyl-2-{3-methyl-2-[4-(1-methyl-piperidin-4-yl)-butoxy]-pyridin-
4-yl}-1H-benzoimidazole;
2-{3-Chloro-2-[4-(1-methyl-piperidin-4-yl)-butoxy]-pyridin-4-yl}-4-methyl-1H-
benzoimidazole;
2-{3-Chloro-2-[4-(1-methyl-piperidin-4-yl)-butoxy]-pyridin-4-yl}-4,5-dimethyl-
1H-benzoimidazole;
4-Chloro-2-{3-chloro-2-[4-(1-methyl-piperidin-4-yl)-butoxy]-pyridin-4-yl}-1H-
benzoimidazole;
2-{3-Chloro-2-[4-(1-methyl-piperidin-4-yl)-butoxy]-pyridin-4-yl}-5-fluoro-4-
methyl-1H-benzoimidazole;
2-{3-Chloro-2-[4-(1-methyl-piperidin-4-yl)-butoxy]-pyridin-4-yl}-4,6-dimethyl-
1H-benzoimidazole;
6-Chloro-2-{3-chloro-2-[4-(1-methyl-piperidin-4-yl)-butoxy]-pyridin-4-yl}-4-
methyl-1H-benzoimidazole;
5-Chloro-2-{3-chloro-2-[4-(1-methyl-piperidin-4-yl)-butoxy]-pyridin-4-yl}-4-
methyl-1H-benzoimidazole;
5-Fluoro-4-methyl-2-{5-methyl-2-[4-(1-methyl-piperidin-4-yl)-butoxy]-pyridin-
4-yl}-1H-benzoimidazole;
5-Chloro-6-fluoro-2-{5-methyl-2-[4-(1-methyl-piperidin-4-yl)-butoxy]-pyridin-
4-yl}-1H-benzoimidazole;
5-tert-Butyl-2-{5-methyl-2-[4-(1-methyl-piperidin-4-yl)-butoxy]-pyridin-4-yl}-
1H-benzoimidazole;
4,5-Dimethyl-2-{5-methyl-2-[4-(1-methyl-piperidin-4-yl)-butoxy]-pyridin-4-yl}-
1H-benzoimidazole;
2-{5-Chloro-2-[4-(1-methyl-piperidin-4-yl)-butoxy]-pyridin-4-yl}-4,6-dimethyl-
1H-benzoimidazole;
5-Chloro-2-{5-chloro-2-[4-(1-methyl-piperidin-4-yl)-butoxy]-pyridin-4-yl}-1H-
benzoimidazole;
5-Chloro-2-{5-chloro-2-[4-(1-methyl-piperidin-4-yl)-butoxy]-pyridin-4-yl}-6-
fluoro-1H-benzoimidazole;
5-tert-Butyl-2-{5-chloro-2-[4-(1-methyl-piperidin-4-yl)-butoxy]-pyridin-4-yl}-
1H-benzoimidazole;
2-{5-Bromo-2-[4-(1-methyl-piperidin-4-yl)-butoxy]-pyridin-4-yl}-4-chloro-1H-
benzoimidazole;
2-{5-Bromo-2-[4-(1-methyl-piperidin-4-yl)-butoxy]-pyridin-4-yl}-5-chloro-6-
fluoro-1H-benzoimidazole;
2-{5-Bromo-2-[4-(1-methyl-piperidin-4-yl)-butoxy]-pyridin-4-yl}-5-chloro-1H-
benzoimidazole;
1-(3-{4-[4,5-Bis-(4-bromo-phenyl)-1H-imidazol-2-yl]-3-chloro-phenoxy}-
propyl)-4-methyl-piperazine;
1-{3-[3-Chloro-4-(4,5-diphenyl-1H-imidazol-2-yl)-phenoxy]-propyl}-4-methyl-
piperazine;
1-(3-{4-[4,5-Bis-(2-chloro-phenyl)-1H-imidazol-2-yl]-3-chloro-phenoxy}-
propyl)-4-methyl-piperazine;
1-(3-{4-[4,5-Bis-(4-methoxy-phenyl)-1H-imidazol-2-yl]-3-chloro-phenoxy}-
propyl)-4-methyl-piperazine;
1-{3-[3-Chloro-4-(4,5-di-p-tolyl-1H-imidazol-2-yl)-phenoxy]-propyl}-4-methyl-
piperazine;
1-(3-{4-[4,5-Bis-(4-fluoro-phenyl)-1H-imidazol-2-yl]-3-chloro-phenoxy}-
propyl)-4-methyl-piperazine;
1-(3-{4-[4,5-Bis-(3-methoxy-phenyl)-1H-imidazol-2-yl]-3-chloro-phenoxy}-
propyl)-4-methyl-piperazine;
1-(3-{4-[4,5-Bis-(3-methoxy-phenyl)-1H-imidazol-2-yl]-2-fluoro-phenoxy}-
propyl)-4-methyl-piperazine;
1-(3-{4-[4,5-Bis-(4-bromo-phenyl)-1H-imidazol-2-yl]-3-chloro-phenoxy}-
propyl)-4-methyl-[1,4]diazepane;
1-(3-{4-[4,5-Bis-(3-methoxy-phenyl)-1H-imidazol-2-yl]-3-chloro-phenoxy}-
propyl)-4-methyl-[1,4]diazepane
1-{3-[2-Chloro-4-(5-methyl-4-phenyl-1H-imidazol-2-yl)-phenoxy]-propyl}-4-
methyl-[1,4]diazepane;
2-{3-Chloro-4-[3-(4-methyl-[1,4]diazepan-1-yl)-propoxy]-phenyl}-4,5,6,7-
tetrahydro-1H-benzoimidazole;
2-{2-Chloro-4-[3-(4-methyl-[1,4]diazepan-1-yl)-propoxy]-phenyl}-4,5,6,7-
tetrahydro-1H-benzoimidazole;
1-Methyl-4-{3-[3-methyl-4-(4-phenyl-5-trifluoromethyl-1H-imidazol-2-yl)-
phenoxy]-propyl}-piperidine;
4-{3-[3-Chloro-4-(4-phenyl-5-trifluoromethyl-1H-imidazol-2-yl)-phenoxy]-
propyl}-1-methyl-piperidine;
4-(3-{3-Chloro-4-[5-methyl-4-(3-trifluoromethyl-phenyl)-1H-imidazol-2-yl]-
phenoxy}-propyl)-1-methyl-piperidine;
4-(3-{3-Chloro-4-[4-(3,5-dichloro-phenyl)-5-methyl-1H-imidazol-2-yl]-
phenoxy}-propyl)-1-methyl-piperidine;
4-(3-{4-[4-(3,5-Dichloro-phenyl)-5-methyl-1H-imidazol-2-yl]-3-methyl-
phenoxy}-propyl)-1-methyl-piperidine;
4-(3-{3-Chloro-4-[4-(4-chloro-phenyl)-5-methyl-1H-imidazol-2-yl]-phenoxy}-
propyl)-1-methyl-piperidine;
4-(3-{4-[4,5-Bis-(4-fluoro-phenyl)-1H-imidazol-2-yl]-3-chloro-phenoxy}-
propyl)-1-methyl-piperidine;
4-(3-{4-[4,5-Bis-(3-methoxy-phenyl)-1H-imidazol-2-yl]-3-chloro-phenoxy}-
propyl)-1-methyl-piperidine;
4-(3-{3-Chloro-4-[4-(4-chloro-phenyl)-5-p-tolyl-1H-imidazol-2-yl]-phenoxy}-
propyl)-1-methyl-piperidine;
2-{2-Chloro-4-[3-(1-methyl-piperidin-4-yl)-propoxy]-phenyl}-4,5,6,7-
tetrahydro-1H-benzoimidazole;
4-{3-[3-Chloro-4-(4-methyl-5-propyl-1H-imidazol-2-yl)-phenoxy]-propyl}-1-
methyl-piperidine;
4-{3-[3-Chloro-4-(5-ethyl-4-methyl-1H-imidazol-2-yl)-phenoxy]-propyl}-1-
methylpiperidine;
1-Methyl-4-(2-{3-methyl-4-[5-methyl-4-(3-trifluoromethyl-phenyl)-1H-
imidazol-2-yl]-phenoxy}-ethoxy)-piperidine;
5-[4-(3,5-Dichloro-phenyl)-5-methyl-1H-imidazol-2-yl]-2-[3-(1-methyl-
piperidin-4-yl)-propoxy]-pyridine;
5-[4-(4-Chloro-phenyl)-5-methyl-1H-imidazol-2-yl]-2-[3-(1-methyl-piperidin-
4-yl)-propoxy]-pyridine;
2-[3-(1-Methyl-piperidin-4-yl)-propoxy]-5-[5-methyl-4-(3-trifluoromethyl-
phenyl)-1H-imidazol-2-yl]-pyridine;
2-[3-(1-Methyl-piperidin-4-yl)-propoxy]-5-[5-methyl-4-(4-trifluoromethyl-
phenyl)-1H-imidazol-2-yl]-pyridine;
2-[3-(1-Methyl-piperidin-4-yl)-propoxy]-5-(4-phenyl-5-trifluoromethyl-1H-
imidazol-2-yl)-pyridine;
1-Methyl-4-(3-{5-[5-methyl-4-(4-trifluoromethyl-phenyl)-1H-imidazol-2-yl]-
pyridin-2-yloxy}-propyl)-piperazine;
1-Methyl-4-(3-{5-[5-methyl-4-(3-trifluoromethyl-phenyl)-1H-imidazol-2-yl]-
pyridin-2-yloxy}-propyl)-piperazine;
4-(4-{3-[4-(4-Chloro-phenyl)-5-methyl-1H-imidazol-2-yl]-phenoxy}-butyl)-1-
methyl-piperidine;
1-Methyl-4-{4-[3-(4-phenyl-5-trifluoromethyl-1H-imidazol-2-yl)-phenoxy]-
butyl}-piperidine;
2-[4-(1-Methyl-piperidin-4-yl)-butoxy]-4-(4-phenyl-5-trifluoromethyl-1H-
imidazol-2-yl)-pyridine;
2-[4-(1-Methyl-piperidin-4-yl)-butoxy]-4-[5-methyl-4-(3-trifluoromethyl-
phenyl)-1H-imidazol-2-yl]-pyridine;
4-{3-[4-(5-Isobutyl-4-methyl-1H-imidazol-2-yl)-3-methyl-phenoxy]-propyl}-
1-methyl-piperidine;
4-[4-(4-Chloro-phenyl)-5-methyl-1H-imidazol-2-yl]-2-[4-(1-methyl-piperidin-
4-yl)-butoxy]-pyridine;
4-{3-[3-Chloro-4-(5-isobutyl-4-methyl-1H-imidazol-2-yl)-phenoxy]-propyl}-
1-methyl-piperidine;
1-Methyl-4-(4-{3-[5-methyl-4-(4-trifluoromethyl-phenyl)-1H-imidazol-2-yl]-
phenoxy}-butyl)-piperidine;
1-{3-[2-Chloro-4-(1H-imidazol-2-yl)-phenoxy]-propyl}-4-methyl-piperazine;
1-{3-[3-Chloro-4-(4,5-dimethyl-1H-imidazol-2-yl)-phenoxy]-propyl}-4-
methyl-piperazine;
1-{3-[3-Chloro-4-(4-phenyl-5-trifluoromethyl-1H-imidazol-2-yl)-phenoxy]-
propyl}-4-methyl-piperazine;
1-{3-[2-Chloro-4-(4-phenyl-5-trifluoromethyl-1H-imidazol-2-yl)-phenoxy]-
propyl}-4-methyl-[1,4]diazepane;
1-Methyl-4-(3-{3-methyl-4-[5-methyl-4-(3-trifluoromethyl-phenyl)-1H-
imidazol-2-yl]-phenoxy}-propyl)-piperidine;
4-(3-{4-[4-(4-Chloro-phenyl)-5-methyl-1H-imidazol-2-yl]-3-methyl-
phenoxy}-propyl)-1-methyl-piperidine;
4-(2-{4-[4-(4-Chloro-phenyl)-5-methyl-1H-imidazol-2-yl]-3-methyl-
phenoxy}-ethoxy)-1-methyl-piperidine;
1-(3-{4-[4-(4-Chloro-phenyl)-5-methyl-1H-imidazol-2-yl]-3-methyl-
phenoxy}-2-methyl-propyl)-4-methyl-piperazine;
2-[4-(4-Chloro-phenyl)-5-methyl-1H-imidazol-2-yl]-6-[4-(1-methyl-piperidin-
4-yl)-butoxy]-pyridine;
4-Methyl-2-[3-(1-methyl-piperidin-4-yl)-propoxy]-5-[5-methyl-4-(3-
trifluoromethyl-phenyl)-1H-imidazol-2-yl]-pyridine;
5-Bromo-4-[4-(4-chloro-phenyl)-5-methyl-1H-imidazol-2-yl]-2-[4-(1-methyl-
piperidin-4-yl)-butoxy]-pyridine;
2,4-Dimethyl-1-{3-[4-(4-phenyl-5-trifluoromethyl-1H-imidazol-2-yl)-
phenoxy]-propyl}-piperazine;
1,2-Dimethyl-4-{3-[4-(4-phenyl-5-trifluoromethyl-1H-imidazol-2-yl)-
phenoxy]-propyl}-piperazine;
3-Chloro-2-[4-(1-methyl-piperidin-4-yl)-butoxy]-4-(4-phenyl-5-
trifluoromethyl-1H-imidazol-2-yl)-pyridine;
1-Methyl-4-(4-{4-[5-methyl-4-(3-trifluoromethyl-phenyl)-1H-imidazol-2-yl]-
pyridin-2-yloxy}-butyl)-[1,4]diazepane;
5-Bromo-2-[4-(1-methyl-piperidin-4-yl)-butoxy]-4-[5-methyl-4-(3-
trifluoromethyl-phenyl)-1H-imidazol-2-yl]-pyridine;
4-[4-(4-Chloro-phenyl)-5-trifluoromethyl-1H-imidazol-2-yl]-2-[4-(1-methyl-
piperidin-4-yl)-butoxy]-pyrimidine;
4-(3-{4-[5-Cyclopropylmethyl-4-(3-trifluoromethyl-phenyl)-1H-imidazol-2-
yl]-3-methyl-phenoxy}-propyl)-1-methyl-piperidine;
1-{4-[4-(4-Chloro-phenyl)-5-methyl-1H-imidazol-2-yl]-3-methyl-phenoxy}-3-
(4-methyl-piperazin-1-yl)-propan-2-ol;
4-(3-{3-Chloro-4-[4-(4-chloro-phenyl)-5-methyl-1H-imidazol-2-yl]-phenoxy}-
propyl)-piperidine;
4-(3-{3-Chloro-4-[4-(4-chloro-phenyl)-5-methyl-1H-imidazol-2-yl]-phenoxy}-
propyl)-1-ethyl-piperidine;
4-(3-{3-Chloro-4-[4-(4-chloro-phenyl)-5-methyl-1H-imidazol-2-yl]-phenoxy}-
propyl)-1-isopropyl-piperidine;
1-Methyl-4-{3-[4-(4-phenyl-5-trifluoromethyl-1H-imidazol-2-yl)-naphthalen-
1-yloxy]-propyl}-piperidine;
1-(4-Methyl-piperazin-1-yl)-3-{5-[5-methyl-4-(4-trifluoromethyl-phenyl)-1H-
imidazol-2-yl]-pyridin-2-yloxy}-propan-1-one;
6-[4-(4-Chloro-phenyl)-5-methyl-1H-imidazol-2-yl]-3-fluoro-2-[4-(1-methyl-
piperidin-4-yl)-butoxy]-pyridine;
1-Methyl-4-(4-{3-methyl-6-[5-methyl-4-(3-trifluoromethyl-phenyl)-1H-
imidazol-2-yl]-pyridin-2-yloxy}-butyl)-piperazine;
1-Methyl-4-{3-[4-(5-methyl-4-thiophen-2-yl-1H-imidazol-2-yl)-phenoxy]-
propyl}-piperidine;
2-{3-[4-(1-Methyl-piperidin-4-yl)-butoxy]-phenyl}-3H-imidazo[4,5-b]pyridine;
(5-Chloro-1H-indol-2-yl)-(4-methyl-piperazin-1-yl)-methanone;
(5-Fluoro-1H-indol-2-yl)-(4-methyl-piperazin-1-yl)-methanone;
(5-Bromo-1H-indol-2-yl)-(4-methyl-piperazin-1-yl)-methanone;
(5-Methyl-1H-indol-2-yl)-(4-methyl-piperazin-1-yl)-methanone;
(5,7-Difluoro-1H-indol-2-yl)-(4-methyl-piperazin-1-yl)-methanone;
(7-Chloro-1H-indol-2-yl)-(4-methyl-piperazin-1-yl)-methanone;
(5,7-Dichloro-1H-indol-2-yl)-(4-methyl-piperazin-1-yl)-methanone; (3,5-
Dichloro-1H-indol-2-yl)-(4-methyl-piperazin-1-yl)-methanone;
(6-Chloro-1H-indol-2-yl)-(4-methyl-piperazin-1-yl)-methanone;
(1H-Indol-2-yl)-(3-methyl-piperazin-1-yl)-methanone;
(7-Bromo-1H-indol-2-yl)-(4-methyl-piperazin-1-yl)-methanone;
(5-Bromo-benzofuran-2-yl)-(4-methyl-piperazin-1-yl)-methanone;
(1H-Indol-2-yl)-(4-methyl-piperazin-1-yl)-methanethione;
[5-(5-Fluoro-4-methyl-1H-benzoimidazol-2-yl)-4-methyl-pyrimidin-2-yl]-[3-
(1-methyl-piperidin-4-yl)-propyl]-amine; and
[5-(4,6-Dimethyl-1H-benzoimidazol-2-yl)-4-methyl-pyrimidin-2-yl]-[3-(1-
methyl-piperidin-4-yl)-propyl]-amine.

A “pharmaceutically acceptable salt” is intended to mean a salt of a free acid or base compound that is non-toxic, biologically tolerable, or otherwise biologically suitable for administration to the subject. See, generally, Berge, et al., “Pharmaceutical Salts”, J. Pharm. Sci., 1977, 66:1-19. Exemplary pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

An antihistaminergic agent used in accordance with the invention may be selective or specific to the H1 or H4 receptor, or it may be non-specific and bind to or inhibit another receptor. Thus, one or more of the antihistaminergic agents of the invention may be a dual inhibitor. For example, a dual H3/H4 inhibitor (see, e.g., US Patent Application Publication No. 2005/0090527) may be used in combination with a centrally acting H1 receptor antagonist.

Optionally, the antihistaminergic agents may be combined with additional active ingredients, including one or more other antihistaminergic agents and/or other active pharmaceutical ingredients. The additional active ingredients may be co-administered separately with the antihistamine agents of the invention (i.e., each in its own unit dosage form), or one or more of the active ingredients may be delivered together in a single composition or unit dosage form containing the antihistamine agents in a pharmaceutical composition according to the invention.

In one embodiment, the centrally acting H1R antagonist and the H4R antagonist are coadministered in separate pharmaceutical formulations. Exemplary formulations comprising centrally acting H1R antagonists include formulations of Benadryl® (containing diphenhydramine hydrochloride), Chlor-Trimetron® (containing chlorpheniramine maleate), Atarax (containing hydroxyzine hydrochloride), and Phenergan® (containing promethazine hydrochloride).

In a preferred embodiment, the two antihistamine agents are formulated together into a single pharmaceutical composition. In such pharmaceutical compositions of the invention, the centrally acting H1R antagonist and H4R antagonist, with optional active ingredients, are combined. In a general aspect, a pharmaceutical composition of the invention therefore comprises: (a) an effective amount of at least one centrally acting histamine H1 receptor antagonist; (b) an effective amount of at least one histamine H4 receptor antagonist; and (c) a pharmaceutically acceptable excipient.

A “pharmaceutically acceptable excipient” refers to a substance that is not toxic, biologically intolerable, or otherwise biologically unsuitable for administration to a subject, such as an inert substance, added to a pharmacological composition or otherwise used as a vehicle, carrier, or diluent to facilitate administration of a pharmaceutical agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the pharmaceutical agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation. Additionally, dosage forms may be prepared as immediate-, timed-, controlled-, or extended-release formulations.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the invention can be provided in the form of tablets or capsules, or as a solution, emulsion, or suspension. To prepare the oral compositions, the agents may be formulated to yield a dosage of, e.g., from about 0.05 to about 100 mg/kg daily, or from about 0.1 to about 50 mg/kg daily, or from about 0.25 to about 50 mg/kg daily.

Oral tablets may include the agent and any other active ingredients mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, the compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent, admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

The combination of antihistaminergic agents may alternatively be administered by inhalation, via nasal or oral route, e.g., in a spray formulation also containing a suitable carrier.

Various formulations for delivering the combination of antihistaminergic agents in accordance with the invention, either in separate compositions or a single composition, may be routinely developed in view of guidance in the art. See, e.g., U.S. Pat. Nos. 4,576,604, 4,673,405, 4,857,330, 5,997,905, and 6,149,943; US Patent Application Publication No. 2005/0232986; and International Publication Nos. WO 2003/035070 and 2000/032173.

The advantages flowing from the combination of antihistaminergic agents of the invention is illustrated by the following examples.

### EXAMPLES

### Background Example

### Comparison of Roles of Histamine Receptors in Itch Response in Mouse Model

The role of histamine H4 receptor in itch responses was evaluated in mice. Initially, a robust itch response was produced in Balb/c mice by injection of the mast cell degranulating agent, Compound 48/80 (Sigma, St. Louis, Mo.)], an agent known to cause mast cells to release various biological amines, leukotrienes, and cytokines that may be involved in itch, including the release of a physiologically relevant dose of histamine into the skin, thereby simulating histamine-induced response observed in diseases such as allergic dermatitis. This response was measured in mice through quantification of the number of scratches observed in a 20-minute period subsequent to Compound 48/80 injection.

When the histamine H4R antagonist 5-chloro-1H-indol-2-yl)-(4-methyl-piperazin-1-yl)-methanone (“H4R Inhibitor 1”), which has the structure

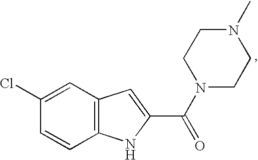

was dosed orally at 30 mg/kg body weight to the mice prior to Compound 48/80 injection, the resulting itch response was reduced by 62.5%. In a further experiment H4R Inhibitor 1 was shown to be significantly active at doses as low as 3 mg/kg, with 12% inhibition observed, and maximally active at 100 mg/kg, with 68% inhibition observed (see FIG. 2).

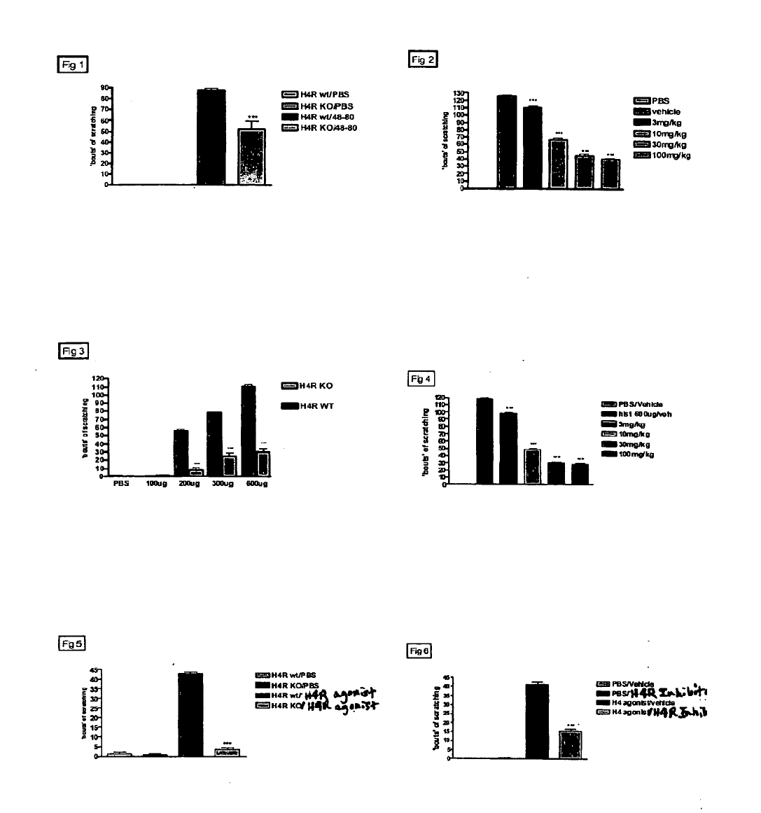

In a further set of experiments, Compound 48/80 was dosed to H4R knockout mice and compared to responses observed in their wild-type littermate controls. A 40% reduction in scratching was observed in the H4R knockout mice compared to the control animals (see FIG. 1).

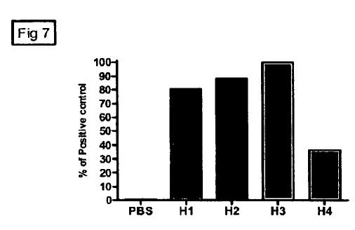

To evaluate the direct role of histamine in the pruritic response, histamine was injected in to the skin of mice and the amount of scratching measured. A robust scratching response was observed, which was dose-dependently inhibited by oral pre-treatment with H4R Inhibitor 1. The inhibition observed was significant at doses as low as 3 mg/kg (17%) and was maximally inhibited at 30 mg/kg (75%) (see FIG. 4). When histamine was injected in to H4R knockout mice, the scratching observed was reduced over that observed in the wild-type littermate controls by 70-85%, dependent on the dose of histamine injected (see FIG. 3).

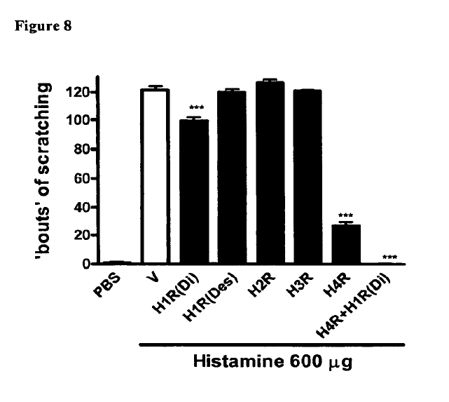

Furthermore, injection of a selective H4R agonist into the skin induced a dose-dependent and significant scratching response that was inhibitable by oral administration of H4R Inhibitor 1 (see FIG. 6) and was absent in H4R knockout mice (see FIG. 5).

As reflected by the experimental results described above, the H4 receptor plays a significant role in pruritic responses mediated by histamine.

In comparison, when certain H1R antagonists were dosed to animals receiving Compound 48/80, the itch response was poorly inhibited (see FIG. 7), as was observed with H2R and H3R antagonists.

### Example 1

### Combination of H4R Antagonist with Centrally Acting H1R Antagonist

a. Histamine-Induced Itch

Methods. Experiments and procedures were performed under on-site IACUC standards and regulations. Female CD-1 mice (Charles River Hollister, Calif.) of approximately 10-12 weeks of age were used. Mice were housed under controlled light (6:00-18:00 h) and temperature (22° C.) with food and water available ad libitum. Histamine diphosphate (Sigma, St. Louis, Mo.) was dissolved in Dulbecco's phosphate buffered saline (PBS, pH=7.4) at various concentrations. H4R Inhibitor 1 was synthesized as described by Jablonowski et al. (J. Med. Chem. (2003), 46, 3957-3960), dissolved in a vehicle of 20% HPBCD/water at various concentrations, and administered per os (p.o.; by mouth) at a volume of 10 ml/kg. Antagonists of other histamine receptors, H1R (diphenhydramine and desloratadine), H2R (ranitidine) and H3R (1-[3-(4-piperidin-1-ylmethyl-phenoxy)-propyl]-piperidine, (“H3R Antagonist 1”; synthesized as described by Barbier et al., British J. Pharmacology (2004), 143: 649-661) were formulated similarly for use in experiments. In all experiments the hair was clipped over the rostral part of the back 24 hours prior to i.d. injection of pruritogen. Where noted, animals received an oral dose of histamine receptor antagonist 20 minutes prior to injection of pruritogen. Animals were injected i.d. with 20 μl of histamine. PBS was given as the vehicle control (“V). Immediately after intradermal injection, the animals were returned to an acrylic cage (approx. 15 cm dia×30 cm) to which they had been acclimated for at least 1 hour prior to the experiment, for observation of itch responses.

Results. Intradermal injection of histamine was found to induce itch in a dose-dependent fashion in CD-1 mice much as it does in humans. Inhibition of histamine H4R by oral dosing of H4R Inhibitor 1 resulted in significant reduction of histamine-induced itch. Small but significant inhibition was demonstrated with diphenhydramine, an H1R antagonist with CNS activity. Combination dosing of diphenhydramine and H4R Inhibitor 1 resulted in total ablation of the pruritic response. In comparison, the administration of the non-centrally acting H1R antagonist desloratadine had no effect. And neither the H2R antagonist ranitidine nor the H3R antagonist demonstrated significant inhibition of histamine-mediated itch. The results are depicted in FIG. 8.

b. Compound 48/80-Induced Itch

Methods. Experiments and procedures were performed under on-site IACUC standards and regulations. Female CD-1 mice (Charles River Hollister, Calif.) of approximately 10-12 weeks of age were used. Mice were housed under controlled light (6:00-18:00 h) and temperature (22° C.) with food and water available ad libitum. Compound 48/80 (Sigma, St. Louis, Mo.) was dissolved in Dulbecco's phosphate buffered saline (PBS, pH=7.4) at 5 mg/ml. H4R Inhibitor 1 (synthesized as previously described) was dissolved in a vehicle of 20% HPBCD/water at various concentrations and administered p.o. at a volume of 10 ml/kg. H1R antagonists diphenhydramine and loratadine were formulated similarly. In all experiments the mouse hair was clipped over the rostral part of the back 24 hours prior to i.d. injection of pruritogen. Where noted, animals received an oral dose of histamine receptor antagonist 20 min prior to injection of pruritogen. Animals were injected i.d. with 20 μl of Compound 48/80 or were given PBS as a vehicle control. Immediately after intradermal injection, the animals were returned to an acrylic cage (approx. 15 cm diameter×30 cm height) to which they had been acclimated for at least 1 h prior to the experiment, for observation of itch responses.

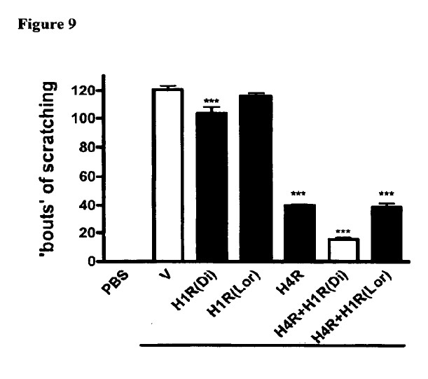

Results. Intra-dermal injection of Compound 48/80 induced itch in a dose-dependent fashion in the CD-1 mice. Inhibition of H4R by oral dosing of H4R Inhibitor 1 significantly reduced the itch. Small but significant inhibition was demonstrated with diphenhydramine. Combination dosing of diphenhydramine and H4R Inhibitor 1 had a synergistic effect in inhibiting the pruritic response. On the other hand, the administration of the non-centrally acting H1R antagonist loratadine had no effect and combination of loratadine with H4R Inhibitor 1 exhibited no additional effects compared to that of H4R Inhibitor 1 alone. The results are illustrated in FIG. 9.

While the invention has been illustrated by reference to examples and preferred embodiments, it is understood that the invention is intended to be not limited by the foregoing detailed description, but defined by the appended claims applying principles of patent law.

What is claimed is:

1. A pharmaceutical composition for treating pruritus, comprising:
an effective amount of at least one centrally acting histamine H1 receptor antagonist;
an effective amount of at least one histamine H4 receptor antagonist; and
a pharmaceutically acceptable excipient.

2. A pharmaceutical composition according to claim 1, wherein said centrally acting histamine H1 receptor antagonist is diphenhydramine, triprolidine, hydroxyzine, pyrilamine, promethazine, or chlorpheniramine, or a pharmaceutically acceptable salt thereof.

3. A pharmaceutical composition according to claim 2, wherein said centrally acting histamine H1 receptor antagonist is diphenhydramine or a pharmaceutically acceptable salt thereof.

4. A pharmaceutical composition according to claim 1, wherein said histamine H4 receptor antagonist is a compound selected from the compounds identified in Table 1 above and pharmaceutically acceptable salts thereof.

5. A pharmaceutical composition according to claim 2, wherein said histamine H4 receptor antagonist is selected from the group consisting of (5-Chloro-1H-indol-2-yl)-(4-methyl-piperazin-1-yl)-methanone, 5-Fluoro-4-methyl-2-{5-methyl-2-[4-(1-methyl-piperidin-4-yl)-butoxy]-pyridin-4-yl}-1H-benzoimidazole, [5-(5-Fluoro-4-methyl-1H-benzoimidazol-2-yl)-4-methyl-pyrimidin-2-yl]-[3-(1-methyl-piperidin-4-yl)-propyl]-amine, and [5-(4,6-Dimethyl-1H-benzoimidazol-2-yl)-4-methyl-pyrimidin-2-yl]-[3-(1-methyl-piperidin-4-yl)-propyl]-amine, and pharmaceutically acceptable salts thereof.

6. A method of treating a subject suffering from pruritus, comprising administering to a subject in need of such treatment: at least one centrally acting histamine H1 receptor antagonist; and an effective amount of at least one histamine H4 receptor antagonist.

7. A method according to claim 6, wherein the centrally histamine H1 receptor antagonist and the histamine H4 receptor antagonist are administered in a single pharmaceutical composition.

8. A method according to claim 6, wherein said histamine H4 receptor antagonist is selected from the group consisting of (5-Chloro-1H-indol-2-yl)-4-methyl-piperazin-1-yl)-methanone, 5-Fluoro-4-methyl-2-{5-methyl-2-[4-(1-methyl-piperidin-4-yl)butoxy]-pyridin-4-yl}-1H-benzoimidazole, [5-(5-Fluoro-4-methyl-1H-benzoimidazol-2-yl)-4-methyl-pyrimidin-2-yl]-[3-(1-methyl-piperidin-4-yl)-propyl]-amine, and [5-(4,6-Dimethyl-1H-benzoimidazol-2-yl)-4-methyl-pyrimidin-2-yl]-[3-(1-methyl-piperidin-4-yl)-propyl]-amine, and pharmaceutically acceptable salts thereof.

9. A method according to claim 8, wherein said centrally acting histamine H1 receptor antagonist is diphenhydramine, triprolidine, hydroxyzine, pyrilamine, promethazine, or chlorpheniramine, or a pharmaceutically acceptable salt thereof.

10. A pharmaceutical composition according to claim 9, wherein said centrally acting histamine H1 receptor antagonist is diphenhydramine or a pharmaceutically acceptable salt thereof.

11. A method according to claim 6, wherein said histamine H4 receptor antagonist is a compound selected from the compounds identified in Table 1 above and pharmaceutically acceptable salts thereof.
